(19) United States
(12) Patent Application Publication
(10) Pub. No.: US 2009/0306584 A1
(43) Pub. Date: Dec. 10, 2009

(54) MEDICAL DEVICE

(76) Inventors: Martin Schmidtlein, Saarbruecken (DE); Thomas Nicola, Spicheren (FR)

Correspondence Address: Muncy, Geissler, Olds & Lowe, PLLC, P.O. BOX 1364, FAIRFAX, VA, 22038-1364, US

(21) Appl. No.: 11/915,374

(22) PCT Filed: May 23, 2006
(86) PCT No.: PCT/EP2006/004881

§ 371 Date: Jun. 1, 2009

(30) Foreign Application Priority Data
May 23, 2005 (DE) 102005024151.4

(51) Int. Cl. A61M 1/28; B29C 65/02

(52) U.S. Cl. 604/28; 604/29; 156/296

(57) ABSTRACT

The invention relates to a medical device which is used, preferably for peritoneal dialysis, and is provided with a film hose.

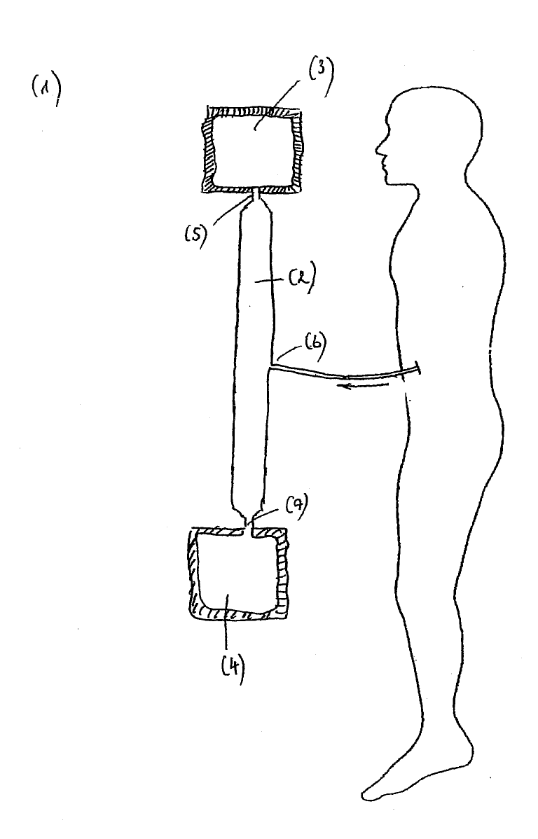

The invention relates to a medical device for removing a liquid from a container or for the filling thereof, a process to be carried out with the device, as well as the use of the device for transferring liquids from a container in the medical sector, specifically for performing peritoneal dialysis.

In the area of medical technology there is often the problem of transferring a liquid, preferably under sterile conditions, from one container to another or the human body, for example in the course of an infusion.

Currently the transfer of liquids in the medical sector is mainly carried out through specific tubes. Such tubes are cost intensive in production, which plays an increasingly larger role in view of the strained financial situation of health systems world wide. Furthermore the mentioned tubes have a tendency for kinking, thereby blocking the tube.

There is therefore a need for a device for the removal of liquids in the medical sector, which overcomes the disadvantages of known systems, i.e., which is inexpensive to produce and is not prone to kinking.

It is an object of the invention to provide such a device.

Therefore one aspect of the invention is a medical device, comprising
(a) a container suitable for holding a liquid
(b) connected thereto a film tube with a connection, and
(c) optionally, one or more further containers suitable for holding a liquid, which are connected with the film tube.

The device according to the invention is cost effective to produce and reduces the problem of kinking. It can be kept in stock in all hospitals without too much in the way of investment costs, particularly in poorer countries.

The containers (a) and optionally (c) are usually bags.

The film tube is a collapsible connecting system.

In the context of the invention, the term “film tube” is to be understood, especially in terms of a distinction from the term “tube”, in that a film tube, in contrast to a tube, shows only restoring forces, which are substantially smaller than its weight force. This means that the film tube collapses without the counter pressure from a filling. By contrast, a tube does have a restoring force which, after compression of the tube, ensures that the tube returns to essentially the original diameter. In addition, the wall thickness of a film tube is smaller than in a tube. In the context of the present invention, the film tube that is to be used according to the invention has a wall thickness preferably of 50 to 300 μm, particularly preferably of 50 to 150 μm, in particular 50 to 120 μm.

The film tube is preferably of such a kind that the inner side does not stick together in case of a heat treatment at temperatures typical for the medical sector, in particular a sterilization temperature of 121° C.

The inner surface of the film tube is therefore generally structured, preferable roughened. Structuring may, e.g., be effected by embossing. Preferably, roughening is effected during extrudation by choosing a suitable jet form.

The roughness can be determined, e.g., with a microscope. Preferred are structures with a depth of roughness R_{z} of 0.05 to 10 μm, preferably 0.1 to 3 μm, in particular 0.1 to 2 μm. In this, the depth of roughness R_{z} is as defined in ISO/DIS 4287/1.

In a preferred embodiment, the film tube is divided into chambers by peel seams and/or valves.

In another preferred embodiment, the connections can be closed depending on the desired use. They can be suitably closed for example by detachable peel seams, valves or clips. The efflux and influx functions are therefore preferably controlled via detachable peel seams, valves or clips.

In a preferred embodiment of the present invention, the container, optionally further containers and connecting film tube of the device are integrally connected to one another.

The join between the container suitable for holding a liquid and the connecting film tube can be produced in any suitable manner according to the invention:

An example that may be mentioned here is that of welding the connecting film tube to the container, in which case the outside of the connecting film tube is welded to the inside of the container, or the inside of the connecting film tube is welded to the container. The connecting film tube can be divided off by peel seams or valves.

Alternatively, the container can also be connected to the film tube via a port element. In this case, the port element can be welded as an intermediate element to the container and to the connecting film tube.

In the context of the invention, a port element is understood as a saddle element, for example. Suitable saddle elements are described in WO 90/06262, whose disclosure in this respect is incorporated by reference into the present invention.

To ensure that liquid does not escape directly after attachment of the device, the connection between container and film tube can initially be closed. In addition to the types of closure already mentioned, this can also suitably be effected by a tear-open seal, such as a peel seam.

In a further embodiment the connecting film tube has a second connection, e.g., to the peritoneum of a patient. This second connection is preferably connected to a catheter, which in turn leads, e.g., into the patient's peritoneum. The catheter is generally an indwelling catheter. The connection provided on the film tube according to the invention for the catheter is preferably designed as a Y-piece or saddle which is fitted onto the film tube. The saddle can be designed in the manner described in WO 90/06262, for example, the disclosure of WO 90/06262 in this respect being incorporated by reference into the present invention.

The Y-piece fitted onto the connecting film tube for attachment to the catheter, or the saddle fitted onto the connecting film tube for attachment to the catheter, should preferably permit the following flow sequences:
(1) Liquid, e.g. a drainage solution, should e.g. be able to flow from a patient's abdominal cavity into an optionally present container, e.g., an empty drainage bag.
(2) Liquid, e.g. a dialysate liquid, should be flushed through the film tube, optionally the saddle or optionally the Y-piece, into the optionally present container, e.g. a drainage bag, in order to clean the line.
(3) Liquid, e.g. a dialysate liquid, should be introduced, e.g. into patient's abdominal cavity.

The saddle or Y-piece should also preferably ensure that no liquid is unnecessarily lost, for example after the device has been detached, e.g., from the patient.

The connecting film tube also optionally has a third connection that can be attached to a further container.

The connecting film tube is connected to a further container, e.g. a drainage bag, preferably in a manner that maintains the sterility conditions.

The join between the container and the connecting film tube can be produced in any suitable manner according to the invention:

An example that may be mentioned here is that of welding the film tube to the container, in which case, depending on the material used for the container and for the film tube, the connecting film tube is welded either to an inside or outside of the container. A peel seam, a valve or a clip is preferably provided for separation from the solution branch.

Alternatively, the container, e.g. a drainage bag, can also be connected to the film tube via a port element, for example a saddle. In this case, the port element can be welded as an intermediate element to the container and to the connecting film tube.

In one embodiment of the present invention, the container for holding a liquid and optional further containers are made of flexible plastic materials. These are preferably co-extruded multi-layer films of plastics that are based on polyolefins.

In a preferred embodiment of the present invention, the containers used for the device according to the invention are free of PVC.

In another preferred embodiment of the present invention, the containers used for the device according to the invention are free of plasticizers.

In another preferred embodiment of the present invention, the containers used for the device according to the invention are free of PVC and plasticizers.

The connecting film tube used to connect the containers comprises one or, preferably, several layers. The multi-layer film tube can be produced in a manner known to the person skilled in the art. For this purpose, a suitable method is, for example, the one described in the German patent application DE-A 103 61 851.

Suitable multi-layer films preferably comprise up to 7 film layers. Multi-layer films with 2 to 5 layers, in particular with 3 layers, are especially preferred.

Materials suitable for the respective layers are, for example, mixtures of modified polypropylene. The polypropylene can be present in mixture with synthetic rubbers, for example SEBS (styrene/ethylene-butylene/styrene), SEPS (styrene/ethylene-propylene/styrene) and SIS (styrene/isoprene/styrene) rubbers. The SEBS and SEPS rubbers are optionally modified by an oil. Copolymers of polyethylene and polypropylene are also preferred.

The phase matrix polymers, in the case of three layer film, generally contain in addition to the polypropylene matrix 10 to 40% by weight of a phase, preferably a block copolymer phase, in the inner layer, 20 to 60% by weight in the middle layer and 0 to 40% by weight in the outer layer.

Film materials having different properties can be obtained through choice of suitable mixtures of the aforementioned plastics in the respective layers.

In a preferred embodiment of the present invention, the connecting film tube is made from at least two layers, the melt temperature of the inner layer and outer layer being set according to the use of the connecting film tube.

In a preferred embodiment the material of the outer layer is a material whose main constituent is a homopolymer of polypropylene whose melting point is preferably about 161° C.

In a further preferred embodiment the outer layer is a sealing layer, comprising a homo- or copolymer based on polypropylene with a melting point of about 125 to 150° C.

If a three-layer film tube structure is present, a polypropylene copolymer preferably functions as middle layer. Alternatively, the middle layer can also comprise a random heterophasic copolymer of polypropylene, preferably comprising one of the above mentioned phases, which is added to the film tube to increase impact resistance. The middle layer preferably has a melting point of 121 to 150° C., particularly preferred 124 to 135° C.

In a preferred embodiment the inner layer of the connecting film tube according to the invention is designed like the middle layer, the inner layer having less impact resistance modifier added to it overall, so that the resulting film tube has better welding properties. The inner layer is preferably made from a material whose main constituent comprises a polypropylene copolymer whose melting point is higher than 135° C.

In a further preferred embodiment the inner layer is a temperature resistant layer comprising a homo- or copolymer based on polypropylene with a melting point of from 125 to 161° C.

Instead of polypropylene the base material may be polyethylene, in which case the materials have a respectively lower melting point.

In a preferred embodiment of the present invention, the connecting film tubes used for the device according to the invention are free of PVC.

In another preferred embodiment of the present invention, the connecting film tubes used for the device according to the invention are free of plasticizers.

In another preferred embodiment of the present invention, the connecting film tubes used for the device according to the invention are free of PVC and plasticizers.

The container (a) and further containers (c), if the latter are present, and the connecting film tube (b) according to the invention can preferably be disposed of jointly after use.

The device according to the invention is preferably produced in the following way:

The device components are provided as film tube or as edge-trimmed film tube in the form of rolled articles. Port elements, for example saddle pieces, are provided as individual parts.

The bag contour or contours are welded non-releasably by heating with a welding device at pressure. The connecting film tube or the port element is then introduced into the bag contour and likewise welded non-releasably to the bag contour.

If a port element is used, it is then connected non-releasably to the connecting film tube by welding.

Peel seams (releasable welds) and/or valves are optionally welded in for dividing the chambers or for defining functions.

It is possible to differentiate or control whether a releasable or non-releasable weld is generated by way of weld temperature, weld pressure and/or weld time.

The connection, e.g. a patient attachment, can be welded non-releasably into or onto the connecting film tube.

The device according to the invention is useful for directing liquids of all kinds in the medical sector, e.g. for the infusion of liquids into the human body. In this case the container (a) is preferably an infusion bag. The liquids are for example blood, blood plasma or an aqueous isotonic solutions with the electrolyxte content of a blood serum, which, e.g., contains drugs or nutrients (e.g. for parenteral feeding.)

Equally preferred the device serves for removing body liquids like blood, urine or wound liquid.

According to the invention the container (a) may be the human body, e.g., when the device is used for haemodialysis, where blood is taken from the patient, is directed through a machine for blood purification and directed back into the body.

In a preferred embodiment the device according to the invention is a device for peritoneal dialysis.

Peritoneal dialysis is a form of artificial washing-out of the blood. In healthy persons, the kidneys filter substances from the blood so that they can be eliminated with the urine. When the kidneys are no longer able to eliminate the products of metabolism, the blood has to be cleaned by artificial means.

Whereas, in the artificial kidney (haemodialysis), the blood is cleaned outside the body by means of a special filter, in peritoneal dialysis the patient's peritoneum, which has a rich supply of blood, is used as an endogenous filter membrane. The peritoneum lines the whole of the abdominal cavity. In peritoneal dialysis, a dialysis solution is allowed to flow several times a day into the abdominal cavity and takes up the poisonous products of metabolism.

In contrast to haemodialysis, peritoneal dialysis is performed alone at home by the patient and can be adapted flexibly to the patient's desired schedule. In the case of peritoneal dialysis, patients are less restricted in terms of their diet and intake of fluids than they are in haemodialysis. However, the catheter placed permanently in the abdominal cavity means there is a risk of infections at the exit site or within the abdominal cavity.

In peritoneal dialysis, the peritoneum is used as blood filter. The peritoneum is a semipermeable membrane which has a rich supply of blood, lines the abdominal cavity and extends over a large number of organs. Dialysis liquid is introduced into the abdominal cavity via a catheter. In this dialysis liquid there is a different concentration of substances than in the blood. In accordance with the principle of osmosis, these substances are withdrawn from the blood and pass into the abdominal cavity. After a few hours, the dialysis liquid is removed again from the abdominal cavity together with the urine constituents.

In peritoneal dialysis, the patient introduces 1.5 to 3.0 litres of a sterile dialysis solution into the abdominal cavity via a catheter, and this solution flushes the peritoneum. The substances to be eliminated migrate from the blood through the peritoneum into the dialysis solution.

A further aim of dialysis is to remove excess water from the body-specialists refer to this as ultrafiltration. Most dialysis solutions therefore contain glucose (sugar). By virtue of a simple osmotic process, water also migrates into the dialysis solution in peritoneal dialysis and can thus be removed too.

After about four to five hours, the dialysis solution, or dialysate, is saturated with poisonous substances. It is withdrawn from the abdominal space via the catheter and replaced by fresh dialysis solution.

Peritoneal dialysis can be performed in a number of different ways. In continuous ambulatory peritoneal dialysis (CAPD), the patients themselves change their dialysis solution about four to five times a day. In automatic peritoneal dialysis (APD), a dialysis device (cycler) ensures automatic bag exchange overnight, so that the patient is more independent during the day and does not feel especially restricted.

Peritoneal dialysis to a large extent corresponds to the natural function of the kidneys since it eliminates poison and water from the body in a continuous and uniform manner. The patient therefore generally has to reckon on fewer side effects. During the dialysis, the patient is mobile and independent and can go about his or her normal activities and work. In the methods for continuous ambulatory peritoneal dialysis, or CAPD, two containers are generally needed to carry out the method. A first container (drainage bag) is provided together with a tube section which can be linked via a catheter to the patient's peritoneum in order to empty the peritoneum. A second container (dialysate bag) contains a dialysate for delivery to the patient's peritoneum. After the liquid has flown from the peritoneum into the drainage bag, the dialysate bag is connected up to the patient. The dialysate is removed from the dialysate bag via the tube and conveyed to the patient's peritoneum. In addition, there are also what are called single-bag systems which have just one dialysate bag and a connecting tube with patient attachment. In this case, after dialysis has been performed, the solution is returned from the peritoneum into the old and emptied dialysate bag. However, after the dialysate has been emptied into the peritoneum, this requires that the peritoneal device remains on the patient for the entire duration of the dialysis. Single bags are also suitable for APD, in which case the cycler disposes of the used dialysate solutions.

Accordingly, the object of the present invention is also to further develop the known devices for peritoneal dialysis in such a way that devices are obtained whose connections between the respective containers and the patient's peritoneum can preferably be produced inexpensively and preferably are not prone to kinking. At the same time, the device should preferably be easy for the patient to handle, so that the dangers of infection of the patient's peritoneum are minimized.

Therefore in a preferred embodiment the device according to the invention is a device for peritoneal dialysis comprising
(a) a dialysate bag (3);
(b) a connecting film tube (2) with patient attachment, and
(c) optionally a drainage bag (4).

The device (1) is then characterized in that the connecting film tube (2) has first (5), second (6) and optionally third (7) connections, the first connection (5) being provided between the connecting film tube (2) and the dialysate bag (3), the peritoneum of a patient being able to be linked via the second connection (6) to the connecting film tube (2), and the optionally present third connection (7) being provided between the optionally present drainage bag (4) and the connecting film tube (2).

Such a device is shown in the FIGURE to which the numerals refer.

The device according to the invention can be used for continuous ambulatory peritoneal dialysis (CAPD) or automatic peritoneal dialysis (APD).

According to the invention, therefore, the tube connections used in known peritoneal devices are replaced by connecting film tubes. Compared to the known tube connections, film tubes are less expensive to produce, so that the resulting device for peritoneal dialysis can be kept in stock in all hospitals without too much in the way of investment costs, particularly in poorer countries. In addition, film tubes are not prone to kinking, which fact ensures simpler and safer handling of the device.

The invention, in a particularly preferred embodiment, thus relates to a device (1) comprising a single-bag or double-bag system for use in performing peritoneal dialysis and having at least the following component parts:
(a) a dialysate bag (3);
(b) a connecting film tube (2) with patient attachment, and
(c) optionally a drainage bag (4),
   in which the connecting film tube (2) connects the dialysate bag (3) via the connection (5) of the film tube (2) and optionally the drainage bag (4) via the optionally present connection (7) of the film tube (2) and comprises a connection (6) for attachment of the patient's peritoneum.

In a further preferred embodiment, the connecting film tube is divided into chambers by peel seams and/or valves.

In another preferred embodiment, the connections (5), (6) and/or (7) can be closed depending on the desired peritoneal dialysis procedure. They can be suitably closed for example by detachable peel seams, valves or clips. The efflux, influx and flush functions are therefore preferably controlled via detachable peel seams, valves or clips.

At the start of a typical cycle of continuous ambulatory peritoneal dialysis, the solution situated in the peritoneal cavity is removed from said peritoneal cavity. If the device according to the invention comprises a drainage bag (4), the solution in the peritoneal cavity is thus conveyed via the catheter and the connecting film tube (2) connected thereto into the drainage bag (4). For this purpose, the connection (5) between the dialysate bag (3) and the film tube (2) is preferably closed. After the drainage, a connection between the dialysate bag (3) and the patient's peritoneal cavity is made possible via the connecting film tube, while at the same time the connection between the peritoneal cavity and the drainage bag (4) is suppressed.

In the new state of the system, i.e. before its use for peritoneal dialysis, the bags (3) and (4), if the latter is present, and also the connecting film tube (2) can be filled with the dialysate.

In a preferred embodiment of the present invention, the dialysate bag (3), optionally the drainage bag (4) and the connecting film tube (2) of the device (1) are integrally connected to one another.

The dialysate bag (3) of the device according to the invention is intended to receive the dialysate. The dialysate can be, for example, a glucose solution for peritoneal dialysis.

The dialysate bag normally has a volume of 500 to 5000 ml, preferably 1000 to 3000 ml, in particular 1250 to 2750 ml. Particularly when the device according to the invention is used on infants, the dialysate bags employed can also be made smaller. Moreover, the dialysate bag (3) can be divided into several chambers, i.e. at least two chambers, in which case valves and/or peel seams are provided between the respective chambers to permit mixing of the compartments before use in cases where solutions are employed that cannot be kept mixed together.

During the peritoneal dialysis, the dialysate bag (3) is suspended from a stand in such a way that the dialysate liquid can flow, under the effect of the hydrostatic pressure, in the direction of the connection (5) of the film tube, which connection (5) lies at the top as a result of the suspension of the dialysate bag. Therefore, the dialysate bag (3) is preferably provided with at least one suspension hole which serves to engage with a stand so that the dialysate bag (3) is arranged in the correct orientation for administration of the dialysate. The dialysate bag (3) itself can additionally have at least one nozzle through which active substances and/or medicaments can be added to the dialysate bag (3) and thus to the dialysate liquid.

The dialysate bag (3) with the dialysate solution is thus linked to the upper connection (5) of the connecting film tube (2). The connecting film tube (2) is connected to the dialysate bag (3) preferably in a manner that maintains sterile conditions, since, in peritoneal dialysis, delivering dialysate solution to the peritoneum in a non-sterile state can otherwise lead to peritonitis.

The join between the dialysate bag (3) and the connecting film tube (2) can be produced in any suitable manner according to the invention:

An example that may be mentioned here is that of welding the connecting film tube (2) to the dialysate bag (3), in which case the outside of the connecting film tube is welded to the inside of the dialysate bag, or the inside of the connecting film tube is welded to the bag. The connecting film tube can be divided off by peel seams or valves.

Alternatively, the dialysate bag (3) can also be connected to the film tube via a port element. In this case, the port element can be welded as an intermediate element to the bag and to the connecting film tube.

To ensure that the dialysate liquid does not flow into the peritoneum directly after attachment of the device to the patient, the connection (5) between drainage bag (3) and film tube (2) can initially be closed. In addition to the types of closure already mentioned, this can also suitably be effected by a tear-open seal, such as a peel seam.

The connecting film tube (2) has a second connection (6) to the peritoneum of a patient. This second connection (6) is preferably connected to a catheter, which in turn leads into the patient's peritoneum. The catheter is generally an indwelling catheter. The connection (6) provided on the film tube (2) according to the invention for the catheter is preferably designed as a Y-piece or saddle which is fitted onto the film tube. The saddle can be designed in the manner described in WO 90/06262, for example, the disclosure of WO 90/06262 in this respect being incorporated by reference into the present invention.

The Y-piece fitted onto the connecting film tube for attachment to the catheter, or the saddle fitted onto the connecting film tube for attachment to the catheter, should preferably permit the following flow sequences:
(1) The drainage solution should be able to flow from the patient's abdominal cavity into the optionally present and empty drainage bag on the ground.
(2) The dialysate liquid should be flushed through the film tube, optionally the saddle or optionally the Y-piece, into the optionally present drainage bag in order to clean the line.
(3) The dialysate liquid should be introduced into the patient's abdominal cavity.

The saddle or Y-piece should also preferably ensure that no liquid is unnecessarily lost after the device has been detached from the patient.

The connecting film tube (2) also optionally has a third connection (7) that can be attached to a drainage bag (4).

This drainage bag (4) usually has the same volume as or a greater volume than the dialysate container since, particularly when using glucose-containing dialysate solutions, water is conveyed from the body into the dialysate during the peritoneal dialysis and thus subsequently into the drainage bag (4).

In a preferred embodiment, the volume of the drainage bag (4) is 1.5 times greater than the volume of the dialysate bag (3).

The connecting film tube (2) is connected to the drainage bag (4) preferably in a manner that maintains the sterility conditions.

The join between the drainage bag (4) and the connecting film tube (2) can be produced in any suitable manner according to the invention:

An example that may be mentioned here is that of welding the film tube (2) to the drainage bag (4), in which case, depending on the material used for the drainage bag (4) and for the film tube (2), the connecting film tube (2) is welded either to an inside or outside of the drainage bag (4). A peel seam, a valve or a clip is preferably provided for separation from the solution branch.

Alternatively, the drainage bag (4) can also be connected to the film tube via a port element, for example a saddle. In this case, the port element can be welded as an intermediate element to the bag and to the connecting film tube.

In one embodiment of the present invention, the container (3) for the dialysate liquid and the drainage bag (4) are made of flexible plastic materials. These are preferably co-extruded multi-layer films of plastics that are based on, optionally modified, polyolefins.

In a preferred embodiment of the present invention, the containers (3) and (4), if the latter is present, used for the device according to the invention are free of PVC.

In another preferred embodiment of the present invention, the containers (3) and (4), if the latter is present, used for the device according to the invention are free of plasticizers.

In another preferred embodiment of the present invention, the containers (3) and (4), if the latter is present, used for the device according to the invention are free of PVC and plasticizers.

In a further preferred embodiment of the invention the device according to the invention is used for the production of a powder-liquid-mixture. In this case the container according to the invention (a), containing a liquid, is connected—preferably by a peel seam—with a further container, preferably a bag, which contains a powder.

According to the invention a mixing of powder and liquid is effected before removal through the film tube.

Equally preferred the powder and liquid containers may be connected with the film tube, so that the liquid is directed through the film tube into the container with the powder. The removal of the powder-liquid-mixture can then be effected through a further connecting device, which is attached to the container or to the film tube.

The device according to the invention has the advantage, that solid and liquid can be packaged separately under sterile conditions (for the solid, e.g., by irradiation), and are brought into contact only immediately before use.

1. A medical device comprising:
(a) a container suitable for holding a liquid;
(b) connected thereto a film tube with a connection, and
(c) optionally, one or more further containers suitable for holding a liquid, which are connected with the film tube.

2. The device for peritoneal dialysis according to claim 1, further comprising
(a) a dialysate bag;
(b) a connecting film tube with patient attachment, and
(c) optionally a drainage bag,
wherein the film tube has first, second and optionally third connections, the first connection being provided between the film tube and the dialysate bag, the peritoneum of a patient being able to be linked via the second connection to the film tube, and the optionally present third connection being provided between the optionally present drainage bag and the film tube.

3. The device according to claim 1, wherein the connecting film tube is divided into chambers by peel seams and/or valves.

4. The device according to claim 1, wherein the container (a), optionally a container (c) and the connecting film tube (b) of the device are integrally connected to one another.

5. The device according to claim 1, wherein the container (a) is divided into at least two chambers, peel seams and/or valves being provided between the respective chambers to permit connection or separation of the chambers.

6. The device according to claim 1, wherein the container (a) additionally has at least one nozzle through which active substances and/or medicaments can be added to the container (a).

7. The device according to claim 1, wherein the optionally present container (c) additionally has at least one nozzle through which some liquid can be removed.

8. The device according to claim 1, wherein port elements are used for the connections.

9. The device according to claim 1, wherein the connecting film tube has up to 7 film layers.

10. The device according to claim 1, wherein the film layers comprise copolymers of polyethylene and polypropylene or modified polypropylene, the polypropylene being present in mixture with synthetic rubbers, in particular styrene/ethylene-butylene/styrene, styrene/ethylene-propylene/styrene, and styrene/isoprene/styrene rubbers.

11. The device according to claim 1, wherein the connecting film tube comprises at least 2 layers, where
the main constituent of the outer layer is a homopolymer of polypropylene whose melting point is approximately 161° C.,
the main constituent of the inner layer is a polypropylene polymer whose melting point is higher than 135° C.

12. The device according to claim 1, wherein the outer side of the connecting film tube (b) and the inner side of the container (a) are welded together.

13. The device according to claim 3, wherein the container (a) is connected with a further container through a peel seam or through the film tube (b) which container comprises a chamber for holding a powder.

14. Use of the device according to claim 1 for peritoneal dialysis.

15. Method for producing a device according claim 1, comprising:
(1) provision of the device components as optionally edge-trimmed film tube in the form of rolled articles and of port elements as individual parts;
(2) production of the bag contour or bag contours by heating at pressure using welding devices;
(3) insertion of the connecting film tube or of the port element into the bag contour(s) and welding to the bag contour(s);
(4) fitting the patient attachment in or on the connecting film tube by welding.
